(19) United States
(12) Patent Application Publication
(10) Pub. No.: US 2014/0243711 A1
(43) Pub. Date: Aug. 28, 2014

(54) INFORMATION-PROCESSING SYSTEM

(71) Applicant: NINTENDO CO., LTD., Kyoto (JP)

(72) Inventors: Tadashi SUGIYAMA, Kyoto (JP); Hiroshi MATSUNAGA, Kyoto (JP); Yugo HAYASHI, Kyoto (JP)

(73) Assignee: NINTENDO CO., LTD., Kyoto (JP)

(21) Appl. No.: 13/975,641

(22) Filed: Aug. 26, 2013

(30) Foreign Application Priority Data
Feb. 25, 2013 (JP) 2013-35007

(51) Int. Cl. A61B 5/11

(52) U.S. Cl. CPC A61B 5/1118
USPC 600/595

(57) ABSTRACT

An exemplary information-processing system includes: a first measuring unit that measures body activity amount of a user; a second measuring unit that measures body activity amount of the user, the second measuring unit being different from the first measuring unit; a calculation unit that executes a predetermined calculation based on at least the body activity amount measured by the first measuring unit and the body activity amount measured by the second measuring unit; and an output unit that outputs information based on a result of the calculation executed by the calculation unit.

### CROSS REFERENCE TO RELATED APPLICATIONS

This application is based on and claims priorities under 35 USC 119 from Japanese patent application No. 2013-35007, which was filed on Feb. 25, 2013.

### FIELD

The technology herein relates to support body activities (exercises, etc.) of a user.

### BACKGROUND AND SUMMARY

There is known in the art a game system that can calculate a body activity amount of a user. When a user is situated on an interface of the game system a load of the user is calculated by a controller and a result of the calculation is displayed to the user.

The present disclosure provides an information-processing system including: a first measuring unit that measures a body activity amount of a user; a second measuring unit that measures a body activity amount of the user, the second measuring unit being different from the first measuring unit; a calculation unit that executes a predetermined calculation based on at least the body activity amount measured by the first measuring unit and the body activity amount measured by the second measuring unit; and an output unit that outputs information based on a result of the calculation executed by the calculation unit.

### BRIEF DESCRIPTION OF THE DRAWINGS

Exemplary embodiments hereafter will be described with reference to the following drawings, wherein:

FIG. 1 shows an exemplary non-limiting a diagram illustrating a whole configuration of a fitness support system;

FIG. 2 shows an exemplary non-limiting a block diagram illustrating a hardware configuration of a terminal device;

FIG. 3 shows an exemplary non-limiting block diagram illustrating a hardware configuration of a main device;

FIG. 4 shows an exemplary non-limiting block diagram illustrating a functional configuration of the fitness support system;

FIG. 5 shows an exemplary non-limiting flow chart illustrating a process of the main device;

FIG. 6 shows an exemplary non-limiting displayed screen;

FIG. 7 shows an exemplary non-limiting displayed screen;

FIG. 8 shows an exemplary non-limiting displayed screen; and

FIG. 9 shows an exemplary non-limiting displayed screen.

### DETAILED DESCRIPTION OF NON-LIMITING EXEMPLARY EMBODIMENTS

### Exemplary Embodiment

### (1) Configuration

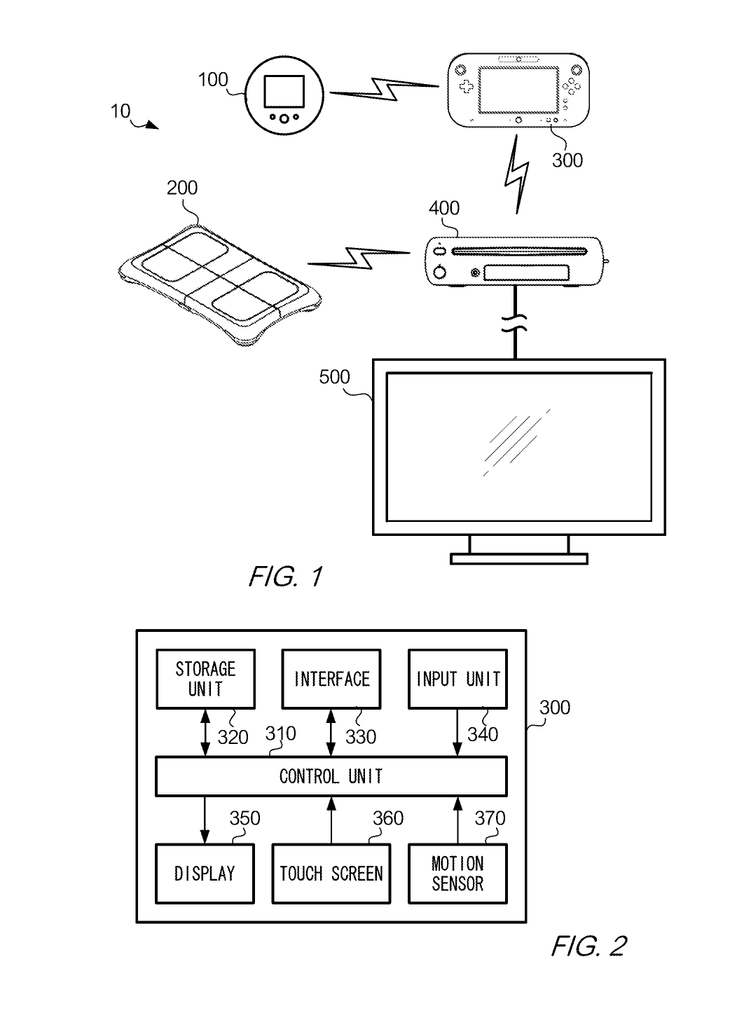

FIG. 1 is a diagram illustrating an entire configuration of fitness support system 10 according to an exemplary embodiment of the present technology. Fitness support system 10 is an information-processing system for measuring a body activity amount of a user and supporting body activities of the user. Fitness support system 10 includes activity meter 100, load input device 200, terminal device 300, main device 400 and display device 500. It is assumed that fitness support system 10 will be set up inside a home of the user; however, activity meter 100 may be used outside of the user's home.

In this application, the term “body activity” refers to any activity of a human body that is associated with consumption of energy. Thus, “body activity” as used herein is not limited to specifically formal exercise; namely to a physical exercise routine with an object of maintaining and/or improving physical strength and stamina. Rather, the term “body activity” as used herein may include, for example, physical exertion expended in climbing up or down stairs, walking, carrying out household tasks, and the like. In the present exemplary embodiment the term “calories (cals)” shall be used to quantify an amount of such body activity. Also, in the present exemplary embodiment, MET (Metabolic Equivalent Task) is used as a unit of exercise intensity value, and thus in measuring body activity an exercise intensity value consisting of quantifiable units is used. For each body activity to be measured, a predetermined value that is equal to or more than “1.0” is provided as an exercise intensity value, wherein a value of “1.0” is equivalent to an exercise intensity of a body at rest.

In the present exemplary embodiment, caloric expenditure resulting from body activity, i.e. an amount of calories burned by a user when performing a body activity, is calculated by multiplying an exercise intensity value of the body activity being performed by the user by a body weight of the user, and by an amount of time for which the body activity is performed, and further by a value of 1.05. It is of note here that calorie consumption of users performing body activities having an equivalent exercise intensity value over an equal period of time is likely to differ among the users depending on their respective body weights. Also, it is of note that calculation of caloric expenditure may be derived by a formula other than the one described above.

Activity meter 100 is a portable measuring device for measuring body activity amounts of a user. Activity meter 100 includes an acceleration sensor, an altitude sensor (a pneumatic sensor), and so on, and transmits measurement data sets indicating measured body activity amounts of the user to terminal device 300. In this example, transmission of measurement data sets from activity meter 100 to terminal device 300 is carried out via infrared communication. However, transmission of such data sets is not limited to infrared, and may be carried out by use of other appropriate wireless communication. Alternatively, activity meter 100 may be wired for communication with terminal device 300 by utilizing, for example, USB (Universal Serial Bus) terminals. Furthermore, activity meter 100 may include a display for displaying body activity amounts, and/or information related to body activity amounts.

Activity meter 100 provides a measurement data set indicative of a body activity amount per unit time (for example, per minute). Measurement data sets of the present exemplary embodiment may include data sets indicative of a number of steps measured by activity meter 100, as well as, for example, data indicative of a change in altitude over time, together with data on body activity and exercise strength. Body activities measured by activity meter 100 preliminarily are classified as one or more of several types of body activity such as “walking,” “climbing up and/or down stairs,” “brisk walking,” “jogging,” and so on, and for each type of body activity an ID may be assigned. Activity meter 100 is able to determine a type of body activity based on, for example, a change in measured acceleration and/or altitude, and to determine a type of body activity being performed at any given time. An ID for a type of body activity is determined in correspondence with an exercise strength required to carry out that body activity.

It is to be noted, however, that data indicative of a body activity amount is not limited to the data described above, and a variety of data for specifying types of body activity may be used. Thus, such data may indicate directly a body activity amount, or may indicate a body activity amount that is determined not directly, but rather by use of a device such as the one described below. For example, activity meter 100 may calculate a body activity amount based on an output value of an acceleration sensor, and so on, and may provide for output the calculated body activity amount as measurement data. Alternatively, activity meter 100 may simply transmit as measurement data a value output from the acceleration sensor, or the like for calculation by main device 400 (or terminal device 300) of a body activity amount based on the transmitted measurement data.

Load input device 200 inputs data based on a load, and is provided with a planar surface where a user is able to place parts of his/her body (hand, foot, bottom, and so on), and a load imposed by a user at the planar surface is detected by use of load sensors such as distortion gauges. These load sensors may be provided at a variety of points at the planar surface, so as to enable the load imposed to be detected relative to a front, rear, left, or right side of the planar surface. Load input device 200 transmits load data indicative of a load detected by one or more of the load sensors. Load input device 200 transmits the load data to main device 400 via Bluetooth (registered trademark). However, as described in relation to transmission of measurement data sets, communication may be carried out by use of any other appropriate communication method.

In the present exemplary embodiment, load input device 200 functions as means for measuring weight (more specifically, as a weight scale), while also functioning as means for assisting measurement of a body activity amount by use of the control program described below. Load input device 200 transmits load data indicative of whether body activity is performed. In this sense, load input device 200 functions as a controller also.

Terminal device 300 is a terminal device for input by a user of an operation to display an image or the like, and may be a portable or hand-held device so that the user can carry out operations while gripping terminal device 300, and operating buttons provided thereon and/or changing an angle of inclination of device 300.

Main device 400 is a computer device that is used for management of an amount of body activity of the user, and causes a program to be executed by which a body activity amount is measured according to operation of the user. Furthermore, main device 400 controls display of images by terminal device 300 and display device 500 in accordance with a result of a user's operation and/or execution of the program. Main device 400 provides display data required for display of images at terminal device 300 and display device 500.

Display device 500 is a standing display device, for example, a television set. Display device 500 may have a display screen larger than that of terminal device 300, and, in the present exemplary embodiment, display device 500 includes such a display screen. Moreover, display device 500 may be a projector used to project images onto a screen or the like.

FIG. 2 is a block diagram illustrating a hardware configuration of terminal device 300. Terminal device 300 comprises control unit 310, storage unit 320, interface 330, input unit 340, display 350, touch screen 360 and motion sensor 370.

Control unit 310 controls operation of each part of terminal device 300, and comprises a processor such as a CPU (Central Processing Unit) and a memory; and causes programs to be executed for performing a variety of processes.

Storage unit 320 is used to store data, and comprises a storage medium such as a flash memory; and stores data used by control unit 310 for controlling processing.

Interface 330 transmits data to and receives data from activity meter 100 and main device 400, via a preferred communication method such as infrared communication. Interface 330 comprises an antenna, a modem, and so on, and communicates with main device 300 via the preferred communication method. For example, interface 330 may communicate with main device 400 via a wireless LAN (Local Area Network).

Input unit 340 receives operations input by a user, and is provided with buttons or the like at predetermined positions and/or may be provided with an analog stick, and provides data indicative of an operation input by the user at control unit 310.

Display 350 displays images. Display 350 comprises a display panel that is composed of a liquid crystal element or an organic EL (electroluminescence) element equipped with a drive circuit, and displays images in accordance with data input.

Touch screen 360 receives an input operation for a screen by the user. Touch screen 360 provides sensors responsive to a display panel of display 350, and provides coordinate data indicating a position (coordinates) indicated by the user to control unit 310. The user can indicate any position on the screen by use of either a finger or a touch pen (stylus).

Motion sensor 370 outputs information indicating movement of terminal device 300. Motion sensor 370 comprises an acceleration sensor (an tri-axial acceleration sensor) for detecting acceleration acting on terminal device 300, a gyro sensor for detecting a change in an angle of inclination, or in an angular velocity of terminal device 300, a geomagnetic sensor for detecting geomagnetism, and so on. Data output by these sensors is transmitted to control unit 310.

It is to be noted that, in the present exemplary embodiment, operation data, coordinate data and sensor data are used for determining operations made by a user. In the following description, unless it is necessary to specify particular ones of these data, the term “operation data” will be used to refer to the data collectively.

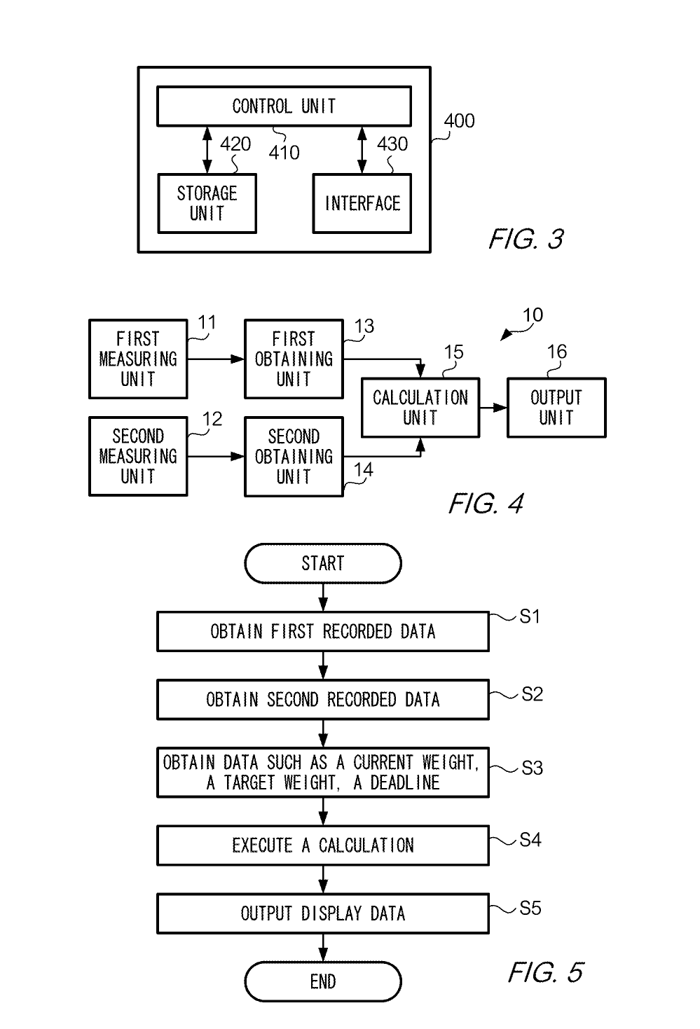

FIG. 3 is a block diagram illustrating a hardware configuration of main device 400. Main device 400 includes control unit 410, storage unit 420 and interface 430.

Control unit 410 controls operation of each part of main device 400, and comprises a processor such as a CPU or a GPU, and a memory. Control unit 410 causes measurement and/or management of body activity to be carried out by executing the control program.

Storage unit 420 stores data, and comprises a storage medium such as a flash memory, a hard disk, and so on. Storage unit 420 may read data on removable storage media such as a memory card or an optical disk, or to record data on the storage media.

Interface 430 transmits data to and receives data from terminal device 300 and display device 500. In the present exemplary embodiment, interface 430 communicates with terminal device 300 wirelessly and communicates with display device 500 by a wired connection. However, communications may be realized by other methods. Furthermore, interface 430 may communicate with external devices (a server device, other fitness support system 10, and so on) via a network such as the Internet.

The hardware configuration of fitness support system 10 is as described above. Fitness support system 10 can manage an amount of body activity of the user and indicate information necessary for the user by the above configuration. When the user inputs his/her current weight, target weight, and a deadline to achieve the target weight, fitness support system 10 can indicate necessary caloric expenditure (target caloric expenditure) to achieve the target weight by the deadline and body activity amount necessary to achieve the target caloric expenditure.

In the present exemplary embodiment, fitness support system 10 measures body activity amount by two methods, and provides information to the user by combining the two methods. A first method is a measuring method using activity meter 100. According to this method, the user can ascertain body activity amount using activity meter 100, by attaching activity meter 100 to a predetermined part of his/her body. Since activity meter 100 is a portable measuring device, the user can measure body activity amount both indoors and outdoors, regardless of location. By the first method, body activity amount by activities such as walking when commuting to office or school, and so on, going upstairs and/or downstairs, and jogging can be measured.

Next, the second method is a measuring method using load input device 200, terminal device 300 and main device 400. According to this method, the user performs a body activity according to an instruction displayed on terminal device 300 or display device 500. In this case, load input device 200 and terminal device 300 are used for measurement of body activity amount secondarily. The body activity in the second method, being different from that of the first method, is assumed to be performed at a predetermined location such as a house of the user. The body activity of the second method is, for example, yoga, a muscular workout and a game (to move the user's body), and is predetermined in the control program.

In the foregoing description, in the second method, individual body activity predetermined in the control program is called “indoor training.” Exercise strength (MET) is set individually for each indoor training activity.

Fitness support system 10 summates body activity amount measured by these methods and indicates information based on the resulting amount. For example, fitness support system 10 can display caloric expenditure of the burned by the first method and the second method, and can display information according to a difference between this caloric expenditure and the target caloric expenditure described above. It is to be noted that “the indication” of information may include not only display (i.e. visual presentation) but also indication by audio.

FIG. 4 is a block diagram showing a functional configuration of fitness support system 10. Being classified according to information indication function, fitness support system 10 comprises first measuring unit 11, second measuring unit 12, first obtaining unit 13, second obtaining unit 14, calculation unit 15, and output unit 16. Functions of second measuring unit 12, first obtaining unit 13, second obtaining unit 14, calculation unit 15, and output unit 16 are realized by main device 400 executing the control program.

First measuring unit 11 measures body activity amount by the first method. As for concrete hardware, activity meter 100 is equivalent to first measuring unit 11. On the other hand, second measuring unit 12 measures body activity amount by the second method. Second measuring unit 12 guides a user in indoor training by animation or a message, and measures body activity amount resulting from the indoor training.

For example, second measuring unit 12 indicates plural indoor training exercises and promotes a choice previously input by the user. When the user chooses any of the indoor training exercises, second measuring unit 12 allows terminal device 300 or display device 500 to display an image promoting practice of the indoor training exercise. For example, when “a push-up” is selected as the indoor training exercise, second measuring unit 12 causes a video and/or a message to be played directing to perform “the push-up” for a predetermined time with the user placing both of his/her hands on load input device 200. Second measuring unit 12 concludes that the user carried out “the push-up” after a predetermined time has passed. Then, second measuring unit 12 calculates the caloric expenditure based on exercise strength set for “the push-up” and time spent exercising by the user.

It is to be noted that second measuring unit 12 may determine whether the user actually performs the indoor training, by sensing whether a load is being applied on load input device 200 (i.e. sensing whether the load data indicating a load greater than a predetermined value was supplied from load input device 200.) In this case, second measuring unit 12 determines the user has not performed the indoor training sufficiently if load indicated by the load data is lower than the predetermined value, and reduces exercise time or caloric expenditure thereof.

In addition, in some indoor training, such a determination may be performed by using terminal device 300 instead of load input device 200. For example, in a case that the user practicing indoor training while holding terminal device 300, second measuring unit 12 can determine whether the user is actually performing the indoor training based on supplied operation data and the value of the data.

Furthermore, second measuring unit 12 may guide the user to switch off activity meter 100 when measuring by the second measuring unit 12. In this way, a problem can be avoided of the user practicing the indoor training while activity meter 100 is mounted whereby caloric expenditure of the indoor training is counted twice.

First obtaining unit 13 obtains measurement data indicating a result of a measurement by first measuring unit 11. Specifically, in the present exemplary embodiment, measurement data measured by activity meter 100 is supplied to main device 400 through terminal device 300. In this regard, activity meter 100 may be constructed such that it can supply the measurement data to main device 400 directly.

Second obtaining unit 14 obtains data indicating a result of a measurement by second measuring unit 12. In the present exemplary embodiment, second obtaining unit 14 obtains, from second measuring unit 12, data indicating caloric expenditure of the body activity measured by second measuring unit 12.

Calculation unit 15 executes a predetermined calculation relevant to body activity amount based on data obtained by first obtaining unit 13 and second obtaining unit 14. Calculation unit 15 can summate the body activity amount measured by first measuring unit 11 and the body activity amount measured by second measuring unit 12, and calculates a resulting value of the body activity. Calculation unit 15 may execute a calculation of a correspondence between the body activity amount measured by first measuring unit 12 and second measuring unit 12 and a predetermined value. For example, calculation unit 15 can calculate the target caloric expenditure by subtracting the summated value of the body activity amount from a predetermined target value.

Output unit 16 outputs information according to a result of a calculation by calculation unit 15. Output unit 16 can output display data to display the summated body activity amount, the body activity amount measured by first measuring unit 11, and the body activity amount measured by second measuring unit 12 in the form of a table and/or a graph. Furthermore, output unit 16 can output display data indicating indoor training necessary to achieve the target caloric expenditure.

### (2) Usage

The user can record his/her weight (current weight) and caloric expenditure and confirm changes therein by using fitness support system 10. Furthermore, by setting the target weight and the deadline, the user can receive a suggestion of indoor training necessary to achieve the target caloric expenditure, namely caloric expenditure necessary to reach the target weight by the deadline. Therefore, the user can achieve the target weight more easily than in a case where no such suggestion is provided. Alternatively, the user can create a personalized indoor training menu and perform a body activity without using such a suggestion.

To receive a suggestion from fitness support system 10, the user needs to input his/her current weight, the target weight and the deadline, in addition to performing a body activity using fitness support system 10. The target weight and the deadline for reaching the target weight may be input only once, and not changed until the user achieves the target weight or the deadline passes. The user can input the target weight and the deadline by operating terminal device 300. On the other hand, the current target may be input repeatedly, such as once a day. The user can measure and input the current target by mounting load input device 200. In this regard, the user may input the current target by operating terminal device 300. Main device 400 records the input current weight, target weight, and deadline in this way.

Furthermore, the user can record his/her body activity using fitness support system 10. For example, the user can record body activity amount when outdoors using activity meter 100, by going out wearing activity meter 100. The user can allow main device 400 to obtain the measurement data recorded by activity meter 100, by having activity meter 100 and terminal device 300 communicate after the user returns home. Furthermore, body activity amount of indoor training that the user performed using load input device 200, and so on, is recorded in main device 400 automatically, without the need for the user to perform any particular operation.

Main device 400 records the body activity amount indicated by measurement data from activity meter 100 (i.e. the body activity amount measured by the first method) and the body activity amount resulting from the indoor training (i.e. the body activity amount measured by the second method) distinctively so as to avoid the two measured amounts being confused. In the following description, for ease of explanation, among the data recorded in main device 400 in the way described above, data indicating the body activity amount measured by the first method is referred to as “first recorded data,” and data indicating the body activity amount measured by the second method is referred to as “second recorded data.”

Main device 400 provides information about body activity amount according to an operation carried out by the user. In the present exemplary embodiment, main device 400 can show a first display to display weight and/or a target caloric expenditure of the user, and a second display to build a menu of indoor training necessary for the user to achieve the target caloric expenditure and to display it. Which display main device 400 shows is determined by an operation carried out by the user.

FIG. 5 is an example of a flow chart illustrating a process regarding display of information by main device 400. At first, control unit 410 of main device 400 reads and obtains the first recorded data and the second recorded data (Step S1, S2.) Each of the first recorded data and the second recorded data is stored in chronological order in a predetermined storage area of storage unit 420. Furthermore, control unit 410 reads and obtains data necessary to display information such as the current weight, target weight, and deadline (Step S3.)

Then, control unit 410 performs a calculation using data obtained in step S1-S3 (Step S4.) Control unit 410 performs a different calculation depending on whether the first display described above is shown or the second display described above is shown. Further, control unit 410 generates and outputs display data according to a result of the calculation in Step S4 (Step S5.) Control unit 410 may supply the display data to terminal device 300, display device 500, or both.

In a case that the first display is shown in Step S4, in addition to summating caloric expenditure indicated by the first recorded data and caloric expenditure indicated by the second recorded data, control unit 410 may calculate a breakdown of each measurement of caloric expenditure according to a predetermined time unit, and summate the caloric expenditure thus calculated. For example, control unit 410 may calculate a breakdown every one hour, every one day, or every one week.

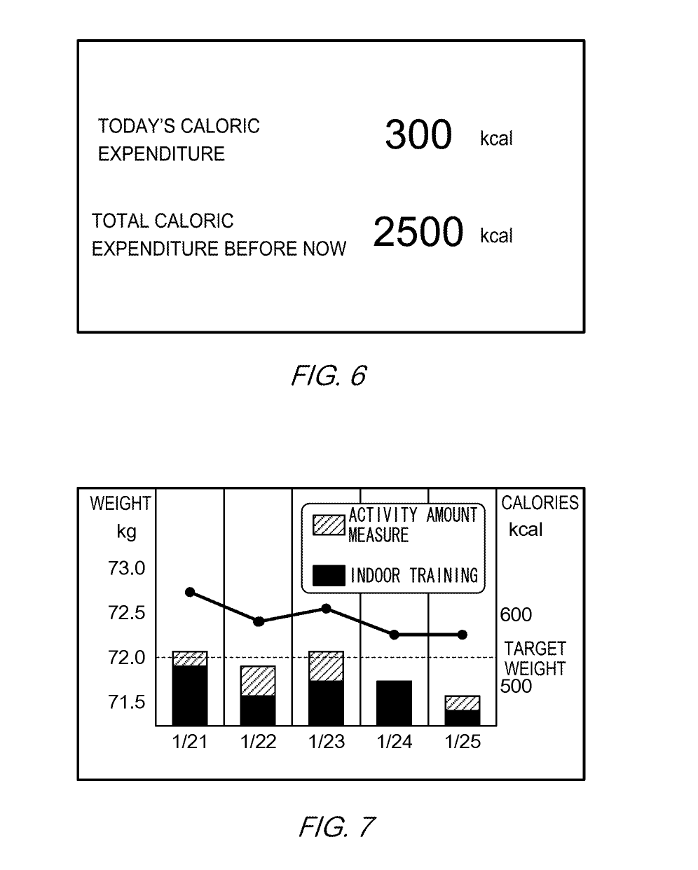

FIG. 6 shows an example of the first display. In the present example, “today's caloric expenditure” indicating the summated value of body activity amount measured during one day and “total caloric expenditure before now” indicating summation of body activity amount from the start of measurement are displayed.

FIG. 7 shows another example of the first display. In the present example, caloric expenditure is displayed by bar graphs, each of the bar graphs corresponding to one day. In addition, each of the bar graphs is displayed in a different color so that the caloric expenditure indicated by the first recorded data and the caloric expenditure indicated by the second recorded data are identifiable. Furthermore, a line graph in the Figure represents a transition of the weight of the user.

It is to be noted that control unit 410 may cause such bar graphs to be displayed every one week instead of every one day, and may allow the user to select a period to be displayed by one bar graph. Furthermore, control unit 410 may cause steps of the user to be displayed instead of the caloric expenditure indicated by the first recorded data, and may cause the caloric expenditure indicated by the second recorded data to be displayed in different colors according to an indoor training exercise performed.

Furthermore, in a case that the second display is performed in Step S4, control unit 410 calculates the target caloric expenditure as follows. First, control unit 410 compares the current weight (the newest weight of the recorded weight) and determines whether the current weight is heavier than the target weight. If the current weight is less than or equal to the target weight, control unit 410 does not set the target caloric expenditure because the user has already attained the target weight.

On the other hand, in a case that the current weight is heavier than the target weight, control unit 410 sets the target caloric expenditure per day by using any of the following expressions (1) to (3) preferentially based on the change in weight of the user. Expression (1) is an expression used in a case when the weight of the user tends to increase during a measurement period (from when the target was set to the present). Expression (2) is an expression used in a case that the weight of the user tends to decrease during the measurement period and it can arrive at the target weight by the deadline if the tendency to decrease weight is consistently maintained. Expression (3) is an expression used in a case that the weight of the user tends to decrease during the measurement period but the target weight cannot be attained by the deadline even if the tendency to decrease weight is consistently maintained.

Cal=[(W−W₀)*7200/D]+Calₐᵥₑ (1)

In expression (1), Cal means the target caloric expenditure per day, and Calₐᵥₑ means an average of the caloric expenditure (the summated value) of the user in the most recent one week (unit in each case is kcal.) W means the current weight and W₀ means the target weight (unit in each case is kg.) D means days until the deadline. In the expression (1), the value of 7200 means calories (kcal) necessary to decrease the weight by 1 kg.

Cal=calₐᵥₑ (2)

In expression (2), the target caloric expenditure per day is equal to the average of the caloric expenditure of the user in the most recent one week. This is because it is more likely that the target weight will be attained by the deadline if a similar body activity is being performed.

Cal=[(W_{d}−W₀)*7200/D]+Calₐᵥₑ (3)

In expression (3), in a case that the weight of the user tends to decrease during the measurement period, W_{d} means estimated weight at the deadline if it is assumed that the weight decrease ratio continues. In comparison with expression (2), the target caloric expenditure per day of expression (3) becomes larger than that of expression (2) due to the first term of the right-hand side; namely [(W_{d}-W₀*7200/D], in contrast, becomes smaller than that of the expression (1).

It is to be noted that control unit 410 does not have to modify the target caloric expenditure each time the current weight changes (i.e. when measuring a user's weight.) For example, a timing to modify the target caloric expenditure is a timing to set the target weight. Timings to set the target weight may include, for example, when measuring user's weight for the first time, when the deadline has passed, and any of the predetermined time units (for example, per one week). Furthermore, the target weight may be set by a predetermined operation performed by the user at any timing.

Control unit 410 may set an upper limit for the target caloric expenditure per day. The upper limit may be a value of suitable calories assumed to be possible to burn per day, or may be determined according to a result of body activities carried out by the user, such as 1.5 times the average of the caloric expenditure of the user in the most recent one week (i.e. Calₐᵥₑ.)

After calculating the target caloric expenditure per day in the way described above, control unit 410 determines whether a body activity has already been carried out on that day. If a body activity has been already carried out by the user, control unit 410 estimates the value by subtracting the caloric expenditure resulting from the body activity to reach the (remaining) target caloric expenditure per day. Control unit 410 uses a value calculated using any of expressions (1) to (3) as the target caloric expenditure per day, if either the first recorded data or the second recorded data of the day has not been recorded.

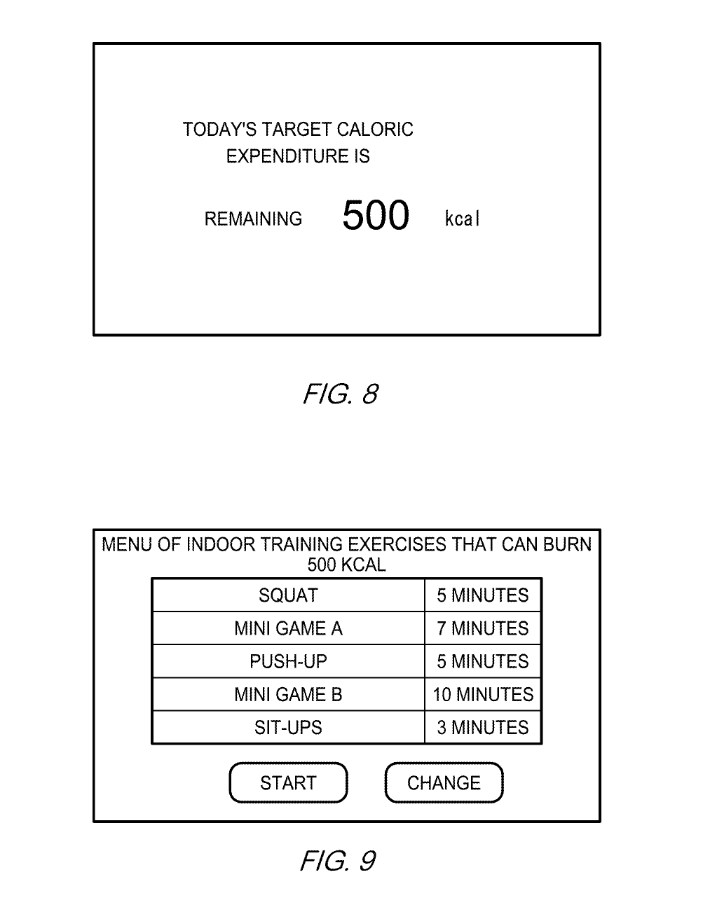

FIG. 8 shows an example of the second display. In this example, the target caloric expenditure per day is indicated by values.

It is to be noted that it is not necessary that the target caloric expenditure, namely the target value of the caloric expenditure, is calculated as values per day. For example, the target caloric expenditure may be calculated as values per week.

FIG. 9 shows another example of the second display. In this example, control unit 410 replaces the target caloric expenditure per day with a specific indoor training menu and causes it to be displayed. In other words, the menu display at that time is one or more indoor training exercise that can burn the target caloric expenditure per day. Control unit 410 calculates caloric expenditure based on the user's current weight, physical strength, and exercise time, for each indoor training exercise, and identifies a combination of indoor training exercises that can achieve the target caloric expenditure per day and presents it to the user. It is to be noted that control unit 410 may prompt the user to select his/her desired combination if there are plural combinations of indoor training exercises that can achieve the target caloric expenditure per day. When the user performs a predetermined operation as shown in the example in FIG. 9, control unit 410 causes an image to be displayed promoting practice of the indoor training exercise and measures the body activity amount.

Control unit 410 may include in the menu body activity measured by activity meter 100 instead of the indoor training exercise or along with the indoor training exercise. Furthermore, control unit 410 may cause different body activities to be displayed in the menu, according to a ratio between the body activity amount measured by the first method (i.e. caloric expenditure measured by activity meter 100) and the body activity amount measured by the second method (i.e. caloric expenditure resulting from the indoor training.) For example, control unit 410 may display a menu promoting practice of indoor training exercises that is suitable for a user who seldom uses activity meter 100. In contrast, control unit 410 may display a menu promoting practice of body activities that can be measured by activity meter 100 so as to promote a use of activity meter 100.

As described above, according to the present exemplary embodiment, the user can receive information based on the result of using in combination the body activity amount measured by activity meter 100 and the body activity amount resulting from the indoor training and summating them. Therefore, the user can obtain information such as caloric expenditure while treating indoor body activities in the same way as outdoor body activities, and it can prevent the body activity used for the calculation of the caloric expenditure being restricted due to location. Furthermore, the user can ascertain the target caloric expenditure according to the actual caloric expenditure and body activities necessary to achieve the target caloric expenditure, to more easily attain the target weight.

In addition, generally, some measuring unit may not be able to measure some body activities appropriately because of problems such as measuring unit not being sufficiently sensitive to measure certain body activities, in a case where there are plural measuring unit to measure the body activity, and measuring methods (including sensors) of each measuring unit are different. For example, if the user performs a body activity such as yoga using very slow movements, most changes in acceleration and/or height do not register in a measuring unit. Therefore, the body activity amount may not be measured appropriately by an acceleration sensor such as activity meter 100. According to the present exemplary embodiment, a measuring method can be targeted to each body activity can be adapted because measurement can be made using the first method or the second method, as appropriate.

### Modifications

The exemplary embodiment described above is one embodiment of implementation of the present technology. The technology is not restricted to the embodiment described above, and can be implemented by embodiments indicated by the following modifications. It is to be noted that the following modifications can be applied in combination, as needed.

### Modification 1

The measuring unit of the present technology is not restricted to first measuring unit 11 and second measuring unit 12 described above. The measuring unit of the present technology may be any means that can measure the body activity. For example, it may be a treadmill or a bicycle-type exercise machine, and may be unit to detect movement of the user by a motion sensor, an image sensor, an infrared ray sensor, and so on, and to measure body activities according to the detected movement. There may be more than three measuring unit.

Display of the present technology may be only one of terminal device 300 and display device 500 described above. In other words, the present technology can be implemented by a constitution in which either terminal device 300 or display device 500 is excluded. Alternatively, terminal device 300 may be an input device (a controller) that does not have display.

Fitness support system 10 of the exemplary embodiment described above is constituted to realize the information-processing device of the present technology by main device 400. However, the information-processing device of the present technology may be provided by terminal device 300 instead of main device 400, and may be such that the constitution equivalent to terminal device 300 and the constitution equivalent to main device 400 are unified. Furthermore, the information-processing device of the present technology may be realized in a server device at least some of whose function is controlled via a network.

The constitution equivalent to main device 400 may be realized by an exclusive computer device, or may be realized by a general-purpose personal computer. Furthermore, main device 400 may be realized by a so-called tablet terminal, a portable game device, a smartphone, and so on. Similarly, a constitution equivalent to activity meter 100 can be realized by a portable game device or a smartphone.

Regarding the present technology, the functional constitution (first obtaining unit 13, second obtaining unit 14, calculation unit 15 and output unit 16) equivalent to the information-processing device may be realized by software. In the case that the information-processing device of the present technology is realized by software, it may be realized by collaboration between plural programs, rather than by a single program (a control program.) In addition, it is not necessary that these programs are executed in the same device, and they may be executed in several devices. It is to be noted that such programs may be provided by a storage medium such as an optical disk or a semiconductor memory, or by download to the information device via a network such as the Internet.

What is claimed is:

1. An information-processing system comprising:
a first measuring unit that measures body activity amount of a user;
a second measuring unit that measures body activity amount of the user, the second measuring unit being different from the first measuring unit;
a calculation unit that executes a predetermined calculation based on at least the body activity amount measured by the first measuring unit and the body activity amount measured by the second measuring unit; and
an output unit that outputs information based on a result of the calculation executed by the calculation unit.

2. The information-processing system according to claim 1, wherein
the first measuring unit measures body activity amount when the user has a portable measuring device, and
the second measuring unit measures body activity amount of a body activity practiced by the user at a predetermined location.

3. The information-processing system according to claim 1, wherein
the calculation unit summates the body activity amount measured by the first measuring unit and the body activity amount measured by the second measuring unit, and
the output unit outputs information for presenting the body activity amount summated by the calculation unit.

4. The information-processing system according to claim 3, wherein
the output unit outputs information for identifiably presenting the body activity amount measured by the first measuring unit and the body activity amount measured by the second measuring unit.

5. The information-processing system according to claim 1, wherein
the output unit outputs information according to body activity amount per a predetermined time unit.

6. The information-processing system according to claim 1, wherein
the calculation unit executes a predetermined calculation based on at least the body activity amount measured by the first measuring unit, the body activity amount measured by the second measuring unit, and a target value of body activity amount set for the user.

7. The information-processing system according to claim 6, wherein
the calculation unit subtracts from the target value the body activity amount measured by the first measuring unit and the body activity amount measured by the second measuring unit.

8. The information-processing system according to claim 6, wherein
the output unit outputs information indicating the body activity amount necessary for the user to reach the target value based on the body activity amount calculated by the calculation unit.

9. The information-processing system according to claim 8, wherein
the information indicates physical exertion.

10. The information-processing system according to claim 9, wherein
the information indicates physical exertion measured by the first measuring unit or the second measuring unit.

11. The information-processing system according to claim 6, wherein
the target value is a predetermined time period calculated based on a target weight of the user and a period for achieving the target weight.

12. A computer-readable non-transitory storage device storing a program causing a computer device to execute:
obtaining first data indicative of body activity amount of a user measured by a first measuring unit;
obtaining second data indicative of body activity amount of a user measured by a second measuring unit being different from the first measuring unit;
executing a predetermined calculation based on at least the first data and the second data; and
outputting information based on a result of the calculation.

13. An information-processing device comprising:
a first obtaining unit that obtains data according to body activity amount of a user measured by a first measuring unit;
a second obtaining unit that obtains data according to body activity amount of the user measured by a second measuring unit different from the first measuring unit;
a calculation unit that executes a predetermined calculation based on at least the data obtained by the first obtaining unit and the second obtaining unit; and
an output unit that outputs information based on a result of the calculation by the calculation unit.

14. An information-processing method performed in a computer device including an input device comprising:
measuring body activity amount of a user by a first measuring unit;
measuring body activity amount of a user by a second measuring unit different from the first measuring unit;
executing a predetermined calculation based on at least the body activity amount measured by the first measuring unit and the body activity amount measured by the second measuring unit; and
outputting information based on a result of the calculation.
